(19) United States
(12) Patent Application Publication
(10) Pub. No.: US 2005/0112723 A1
(43) Pub. Date: May 26, 2005

(54) Mammalian cell lines specifically deficient in O-linked glycosylation

(76) Inventors: Judith L. Fridovich-Keil, Decatur, GA (US); Monty Krieger, Needham, MA (US); Hazel M. Holden, Fitchburg, WI (US); James B. Thoden, Madison, WI (US); Jenny Michele Schulz, Atlanta, GA (US)

Correspondence Address: Christopher B. Linder, Ph.D.;THOMAS, KAYDEN, HORSTEMEYER, & RISLEY, L.L.P., 100 Galleria Parkway, N.W., Suite 1750, Atlanta, GA, 30339-5948, US

(21) Appl. No.: 10/781,012

(22) Filed: Feb. 18, 2004

Related U.S. Application Data
(60) Provisional application No. 60/455,365, filed on Mar. 17, 2003.

(51) Int. Cl. C07H 21/04; C12N 9/54; C12N 1/18; C12N 15/74

(52) U.S. Cl. 435/69.1; 435/221; 435/254.2; 435/254.23; 435/483; 536/23.2

(57) ABSTRACT

Genetically modified cell lines that express a UDP-galactose 4-epimerase (GALE) capable of interconverting UDP-galactose (UDP-gal) and UDP-glucose (UDP-glc), but essentially incapable of interconverting UDP-N-acetylgalactosamine (UDP-galNAc) and UDP-N-acetylglucosamine (UDP-glcNAc).

### CROSS-REFERENCE TO RELATED APPLICATION

This application claims priority to copending U.S. provisional application entitled, “MAMMALIAN CELL LINES SPECIFICALLY DEFICIENT IN O-LINKED GLYCOSYLATION,” having Ser. No. 60/455,365, filed Mar. 17, 2003, which is entirely incorporated herein by reference.

### STATEMENT REGARDING FEDERALLY SPONSORED RESEARCH OR DEVELOPMENT

The U.S. government has a paid-up license in this disclosure and the right in limited circumstances to require the patent owner to license others on reasonable terms as provided for by the terms of DK46403 awarded by the National Institutes of Health (NIH) of the U.S.

### TECHNICAL FIELD

The present disclosure is generally related to providing a means for studying N- and O-linked glycosylation and providing a mammalian cell host capable of producing novel glycoproteins. More particularly, this disclosure is related to genetically modifying a cell line to express UDP-galactose 4-epimerase (GALE) capable of interconverting UDP-galactose (UDP-gal) and UDP-glucose (UDP-glc), but essentially incapable of interconverting UDP-N-acetylgalactosamine (UDP-galNAc) and UDP-N-acetylglucosamine (UDP-glcNAc).

### BACKGROUND

Galactosemia is a rare genetic metabolic disorder. Symptoms of galactosemia are exhibited by elevated blood galactose levels, which may result in mental deficiencies and the formation of cataracts, among other complications, and, if untreated, ultimately death. Most of these symptoms can be avoided with early detection of the disease in children. Relief is given by simply restricting galactose from the diet. Because of the lack of certain enzymes, galactokinase (GALK), galactose- 1 -phosphate uridyl transferease (GALT), or UDP-galactose 4-epimerase (GALE), the body is unable to break down galactose, which then builds up, together with its by-products, and becomes toxic. GALE is the third enzyme in the metabolism of dietary galactose and the key enzyme in de novo synthesis of galactose and its metabolites from glucose. Human GALE catalyzes reversible reactions between UDP-gal and UDP-glc and between UDP-galNAc and UDP-glcNAc. A deficiency of this enzyme results in epimerase deficiency galactosemia, a variant form of galactosemia with clinical severity that ranges from apparantly benign to potentially lethal.

Human GALE catalyzes, as mentioned above, the interconversion of UDP-gal and UDP-glc and the interconversion of UDP-galNAc and UDP-glcNAc. It is known that by interconverting UDP-gal and UDP-glc, GALE activity serves as an important regulator of these metabolite pools, which in turn serve as substrate pools of glucose and galactose for the addition to growing sugar chains for both N-linked and O-linked glycosylation and lipid-linked sugars. It is also known that UDP-galNAc is the obligate first sugar donor for all O-linked glycosylation reactions in mammals. By inhibiting the UDP-galNAc/UDP-glcNAc interconversion, but not UDP-gal/UDP-glc interconversion, glycosylation of N-linked sites can proceed as normal. Glycosylation sites on proteins are classified into two groups—as either N-linked or O-linked. Some glycoproteins carry only N-linked sugars, some carry only O-linked sugars, and many carry both. More than half of all eukaryotic proteins carry covalently attached oligosaccharide or polysaccharide chains.

In N-linked glycoproteins, the glycans are usually attached through N-acetylglucosamine or N-acetylgalactosamine to the side chain amino group in an asparagine residue. In O-linked glycoproteins, glycans are usually attached through an O-glycosidic bond between N-acetylgalactosamine and the hydroxyl group of a threonine or serine residue. Important N-linked glycans are found in ovalbumin and the immunoglobulins. Every immunoglobulin has carbohydrate attached to the constant domain of each heavy chain. Part of the recognition of immunoglobulins is due to the sequence of the oligosaccharide chains of the glycans.

A very important further use of N-linked oligosaccharides is in intracellular targeting in eukaryotic organisms. Proteins destined for certain organelles or for excretion from the cell are marked specifically by oligosaccharides during post translational processing to ensure they arrive at their proper destinations.

Important O-linked glycans appear to function in intracellular targeting and molecular and cellular identification. One example is found in the blood group antigens. Also, mucins, which are found extensively in salivary secretions, contain many short O-linked glycans. These glycoproteins increase the viscosity of the fluids in which they are dissolved.

The bacterial counterpart form of GALE, in particular that from Escherchia coli (E. coli) (WTeGALE), can only interconvert UDP-Gal and UDP-Glc. As discussed above, when the UDP-galNAc and UDP-glcNAc interconversion is absent, and in the absence of environmental sources of UDP-galNAc, glycosylation proceeds via the N-linked pathway only.

Clone ldlD cells are a CHO-derived line originally isolated from a screen for mutants defective in the endocytosis of low density lipoprotein (LDL) as described by Krieger, M. et al. J. Mol.Biol. 150:167-184 (1981). Subsequent studies demonstrated that the LDL receptor defect in these cells was part of a pleiotropic defect in the addition of sugars to glycolipids and glycoproteins, including the LDL receptor, and that these defects all resulted from a loss of GALE activity. Kingsley et al. Cell 44: 749-759(1986); Kingsley et al. The New Eng. J. of Med. 314: 1257-1258(1986). Further, studies by Krieger et al.(1986) and Krieger et al. Methods in Cell Biology 32: 57-84(1989) have demonstrated that the LDL receptor defect, like other glycoprotein and glycolipid defects in ldlD cells, was “environmentally reversible,” meaning that both glycosylation and function could be restored by the addition of low levels of both galactose and galNAc to the culture medium, thereby enabling cellular production of UDP-gal and UDP-galNAc via the sugar salvage pathway. Addition of either gal or galNAc alone enabled only partial glycosylation of the LDL receptor, presumably because, while UDP-gal serves as a galactose donor for the growth of both N- and O-linked sugar chains, UDP-galNAc is the obligate first sugar donor for all O-linked glycosylation in mammals Krieger et al.(1989). Considering that no truly GALE-null patients have been identified, and no GALE mouse knock-out is yet available, ldlD represents the only mammalian cell line currently available that is completely deficient in GALE activity.

Although for over a decade the ldlD cell system has provided a valuable tool for the study of both N- and O-linked glycoproteins in mammalian cells (Krieger et al.(1989)), a fundamental problem has remained—namely that because ldlD cells lack epimerase activity, galactose is not only necessary for their production of UDPgal, it is also toxic to them. Indeed, it was reported that ldlD cells exposed to concentrations of galactose greater than 75 microMolar (μM) will experience toxicity, although wild-type CHO cells demonstrate no apparent toxicity from exposure to galactose levels as high as 10 milliMolar (mM). Krieger et al.(1989). While short-term experiments involving low levels of galactose/galNAc addition are feasible, the biochemical phenotype observed is nonetheless a composite of corrected glycosylation defects superimposed upon metabolic abnormalities resulting from impaired metabolism of galactose. As such, these cells may serve as a useful model system representing epimerase deficiency galactosemia in its most extreme theoretical form, but they cannot support clean dissection of the cellular phenotypes reflecting impaired glycosylation, from those that result from impaired Leloir metabolism of galactose.

Tunicamycin is a known antibiotic that inhibits the synthesis of all N-linked glycoproteins by blocking the transfer of N-acetylglucosamine moiety to dolichol phosphate. The treatment of various cell lines with tunicamycin has permitted the study of glycosylation as it proceeds solely via O-linked glycosylation. There currently exists no counterpart to tunicamycin and no clean mechanism whereby O-linked glycosylation is specifically inhibited to permit the study of N-linked glycosylation in the absence of O-linked glycosylation.

Thus, a heretofore unaddressed need exists in the industry to address the aforementioned deficiencies and/or inadequacies.

### SUMMARY

This disclosure provides an isolated polynucleotide comprising a polynucleotide selected from: a polynucleotide sequence set forth in SEQ ID NO: 1(C307YhGALE) or a degenerate variant of the SEQ ID NO: 1; a polynucleotide sequence at least 90% identical to the polynucleotide sequence set forth in SEQ ID NO: 1; a polynucleotide sequence at least 75% identical to the polynucleotide sequence set forth in SEQ ID NO: 1; and a polynucleotide sequence at least 50% identical to the polynucleotide sequence set forth in SEQ IDNo: 1

Briefly described, SEQ ID NO: 1 is human GALE (hGALE) having an adenine substituted for guanine, changing a TGT codon at residue 307 (encoding cysteine) to a TAT codon (encoding tyrosine) which is identified as C307Y.

The polypeptide of the present disclosure is selected from: an amino acid sequence set forth in SEQ ID NO: 2 (C307YhGALE), or conservatively modified variants thereof; an amino acid sequence that is at least 90% identical to SEQ ID NO: 2; an amino acid sequence that is at least 75% identical to SEQ ID NO: 2; and an amino acid sequence that is at least 50% identical to SEQ ID NO: 2. SEQ ID NO: 2 corresponds to wild type hGALE except a tyrosine residue has been substituted for cysteine at position 307. This single amino acid substitution results in a substantial decrease in the ability of hGALE to interconvert UDP-galNAc and UDP-glcNAc while still maintaining the ability to interconvert UDPgal and UDPglc. It will be appreciated that the substitution of other bulky amino acids, such as phenylalanine, tryptophan or histidine in place of tyrosine as described above may also accomplish the desired results.

The present disclosure further provides a vector comprising the polynucleotide as described above where the vector is preferably pPIC3.5K.

The present disclosure further provides a host cell comprising a vector comprising the polynucleotide described above where the host cell can be Pichia pastoris, Saccharomyces cerevisiae, Schizosaccharomyces pombe, or Escherichia coli. The host cell is preferably Pichia pastoris.

The present disclosure further provides a process for producing a polypeptide comprising culturing a host cell, preferably Pichia pastoris, under conditions sufficient for the production of the polypeptide where the polypeptide has the characteristics that the polypeptide is capable of UDP-gal/UDP-glc interconversion and substantially incapable of UDP-galNAc/UDP-glcNAc interconversion. The polypeptide is selected from: an amino acid sequence set forth in SEQ ID NO: 2 (C307YhGALE) or conservatively modified variants thereof; an amino acid sequence that is at least 90% identical to SEQ ID NO: 2; an amino acid sequence that is at least 75% identical to SEQ ID NO: 2; and an amino acid sequence that is at least 50% identical to SEQ ID NO:2.

The present disclosure further provides a cell line transfected with an expression vector comprising a polynucleotide SEQ ID NO: 1 (C307YhGALE) or a degenerate variant of the SEQ ID NO: 1; a polynucleotide sequence at least 90% identical to the polynucleotide sequence set forth in SEQ ID NO: 1; a polynucleotide sequence at least 75% identical to the polynucleotide sequence set forth in SEQ ID NO: 1; and a polynucleotide sequence at least 50% identical to the polynucleotide sequence set forth in SEQ ID No: 1, encoding a polypeptide having the characteristics that the polypeptide is capable of UDP-gal/UDP-glc interconversion and substantially incapable of UDP-galNAc/UDP-glcNAc interconversion. The polypeptide is selected from: an amino acid sequence set forth in SEQ ID NO: 2 (C307YhGALE), or conservatively modified variants thereof; an amino acid sequence that is at least 90% identical to SEQ ID NO: 2; an amino acid sequence that is at least 75% identical to SEQ ID NO: 2; and an amino acid sequence that is at least 50% identical to SEQ ID NO: 2. The expression vector of the cell line is preferably pCDNA3. The cell line is GALE deficient, preferably ldlD.

The present disclosure further provides a vector comprising an isolated polynucleotide selected from: a polynucleotide sequence set forth in SEQ ID NO: 3 (WTeGALE), or a degenerate variant of the SEQ ID NO: 3; a polynucleotide sequence at least 90% identical to the polynucleotide sequence set forth in SEQ ID NO: 3; a polynucleotide sequence at least 75% identical to the polynucleotide sequence set forth in SEQ ID NO: 3; and a polynucleotide sequence at least 50% identical to the polynucleotide sequence set forth in SEQ ID NO: 3. The vector is preferably pPIC3.5K.

The present disclosure further provides a process for producing a polypeptide comprising culturing a host cell, where the host cell can be Pichia pastoris, Saccharomyces cerevisiae, Schizosaccharomyces pombe, or Escherichia coli, preferably Pichia pastoris, under conditions sufficient for the production of the polypeptide where the polypeptide has the characteristics that the polypeptide is capable of UDP-gal/UDP-glc interconversion and substantially incapable of UDP-galNAc/UDP-glcNAc interconversion. The polypeptide is selected from: an amino acid sequence set forth in SEQ ID NO: 4 (WTeGALE), or conservatively modified variants thereof; an amino acid sequence that is at least 90% identical to SEQ ID NO: 4; an amino acid sequence that is at least 75% identical to SEQ ID NO: 4; and an amino acid sequence that is at least 50% identical to SEQ ID NO: 4.

The present disclosure further provides a cell line transfected with an expression vector comprising a polynucleotide SEQ ID NO: 3 (WTeGALE) or a degenerate variant of the SEQ ID NO: 3; a polynucleotide sequence at least 90% identical to the polynucleotide sequence set forth in SEQ ID NO: 3; a polynucleotide sequence at least 75% identical to the polynucleotide sequence set forth in SEQ ID NO: 3; and a polynucleotide sequence at least 50% identical to the polynucleotide sequence set forth in SEQ ID No: 3, encoding a polypeptide having the characteristics that the polypeptide is capable of UDP-gaVUDP-glc interconversion and substantially incapable of UDP-galNAc/UDP-glcNAc interconversion. The polypeptide is selected from: an amino acid sequence set forth in SEQ ID NO: 4 (WTeGALE), or conservatively modified variants thereof; an amino acid sequence that is at least 90% identical to SEQ ID NO: 4; an amino acid sequence that is at least 75% identical to SEQ ID NO: 4; and an amino acid sequence that is at least 50% identical to SEQ ID NO: 4. The expression vector of the cell line is preferably pCDNA3. The cell line is GALE deficient, preferably ldlD.

The present disclosure further provides a method of culturing a GALE deficient cell line transfected with either a polynucleotide selected from: a polynucleotide sequence set forth in SEQ ID NO: 1 (C307YhGALE) or a degenerate variant of the SEQ ID No: 1; a polynucleotide sequence at least 90% identical to the polynucleotide sequence set forth in SEQ ID NO: 1, a polynucleotide sequence at least 75% identical to the polynucleotide sequence set forth in SEQ ID No: 1, and a polynucleotide sequence at least 50% identical to the polynucleotide sequence set forth in SEQ ID NO: 1 or a polynucleotide selected from: a polynucleotide sequence set forth in SEQ ID NO: 3 (WTeGALE), or a degenerate variant of the SEQ ID NO: 3; a polynucleotide sequence at least 90% identical to the polynucleotide sequence set forth in SEQ ID NO: 3; a polynucleotide sequence at least 75% identical to the polynucleotide sequence set forth in SEQ ID NO: 3; and a polynucleotide sequence at least 50% identical to the polynucleotide sequence set forth in SEQ ID NO: 3 in the absence of galactose to produce glycoproteins having intact N-linked modifications with substantially no O-linked modifications.

Other systems, methods, features, and advantages of the present disclosure will be or will become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure.

FIG. 1 is a comparative illustration of epimerase activity in the purified enzymes wild type human GALE (WThGALE), wild-type E. coli GALE (WTeGALE), and the mutant human enzyme C307YhGALE, with regard to both UDP-gal/UDP-glc interconversion and UDP-galNAc/UDP-glcNAc interconversion.

FIG. 2 is a western blot showing that C307YhGALE (4OkDa band evident in lane 4) can be stably expressed in ldlD cells.

FIG. 3 demonstrates that C307YhGALE expressed in ldlD cells is active with regard to UDP-gal/UDP-glc interconversion.

FIG. 4 is a western blot showing that WTeGALE can be stably expressed in ldlD cells (40 kDa band evident in lane 4).

FIG. 5 demonstrates that WTeGALE expressed in ldlD cells is active with regard to UDP-gal/UDP-glc interconversion.

FIG. 6 demonstrates that C307Y hGALE and WTeGALE in ldlD cells are not significantly active with regard to UDP-galNAc/UDP-glcNAc interconversion, although the WThGALE enzyme in these cells is very active with regard to this reaction.

### DETAILED DESCRIPTION

Polynucleotides, polypeptides, host cells, cell lines and corresponding methods that can be used to study glycosylation or to prepare glycoproteins with novel glycosylation patterns as disclosed.

Prior to setting forth embodiments of the disclosure in detail, it may be helpful to first define the following terms

The term “affinity tag” is used herein to denote a polypeptide segment that can be attached to a second polypeptide (making a fusion protein) to provide for detection of the fusion protein using a monoclonal antibody that recognizes the affinity tag, or purification of the fusion protein using an affinity column of immobilized antibody or other specific ligand (nickel, GST, etc.). In principal, any peptide or protein for which an antibody or other specific binding agent is available can be used as an affinity tag. Affinity tags include HA (a 9 amino acid sequence, derived from the hemagglutinin sequence (tyr-pro-tyr-asp-val-pro-asp-tyr ala), poly-histidine tract (hexahistidine), protein A (Nilsson, et al., EMBO J, 4:1075, 1985; Nilsson, et al., Methods Enzymol., 198:3, 1991), glutathione S transferase (Smith, et al., Gene, 67:31, 1988), Glu-Glu affinity tag, substance P, Flag™ peptide (Hopp, et al., Biotechnology, 6:1204-10, 1988), streptavidin binding peptide, or other antigenic epitope or binding domain. See, in general, Ford, et al., Protein Expression and Purification, 2: 95-107, 1991. DNAs encoding affinity tags are available from commercial suppliers (e.g., Pharmacia Biotech, Piscataway, N.J.).

“Polynucleotide” generally refers to any polyribonucleotide or polydeoxribonucleotide, which may be unmodified ribonucleic acid (RNA) or deoxyribonucleic acid (DNA) or modified RNA or DNA. “Polynucleotides” include, without limitation, single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide“embraces chemically, enzymatically, or metabolically modified forms of polynucleotides as typically found in nature, as well as the chemical forms of DNA and RNA characteristic of viruses and cells. “Polynucleotide” also embraces relatively short polynucleotides, often referred to as oligonucleotides. “Polypeptide” refers to any peptide or protein comprising two or more amino acids joined to each other by peptide bonds or modified peptide bonds, (i.e., peptide isosteres). “Polypeptide” refers to both short chains, commonly referred to as peptides, oligopeptides, or oligomers, and to longer chains, generally referred to as proteins. “Polypeptides” may contain amino acids other than the 20 gene-encoded amino acids. “Polypeptides” include amino acid sequences modified either by natural processes, such as post-translational processing, or by chemical modification techniques, which are well known in the art. Such modifications are described in basic texts and in more detailed monographs, as well as in a voluminous research literature.

Modifications may occur anywhere in a polypeptide, including the peptide backbone, the amino acid side-chains and the amino or carboxyl termini. It will be appreciated that the same type of modification may be present to the same or varying degrees at several sites in a given polypeptide. Also, a given polypeptide may contain many types of modifications. Polypeptides may be branched as a result of ubiquitination, and they may be cyclic, with or without branching. Cyclic, branched, and branched cyclic polypeptides may result from post-translational natural processes, or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination (Proteins—Structure and Molecular Properties, 2nd Ed., T. E. Creighton, W. H. Freeman and Company, New York, 1993; Wold, F., Post-translational Protein Modifications: Perspectives and Prospects, pgs. 1-12 in Post-translational Covalent Modification of Proteins, B. C. Johnson, Ed., Academic Press, New York, 1983; Seifter, et al., Meth Enzymol, 182: 626-646, 1990, and Rattan, et al., Ann NY Acad. Sci., 663:48-62, 1992).

“Variant” refers to a polynucleotide or polypeptide that differs from a reference polynucleotide or polypeptide, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions, and truncations in the polypeptide encoded by the reference sequence, as discussed below.

A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and deletions in any combination. A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques or by direct synthesis.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polynucleotides and polypeptides of the present disclosure.

By way of example, a polynucleotide sequence of the present disclosure may be identical to the reference sequence of SEQ ID NO: 1, that is be 100% identical, or it may include up to a certain integer number of nucleotide alterations as compared to the reference sequence. Such alterations are selected from the group including at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the reference nucleotide by the numerical percent of the respective percent identity (divided by 100) and subtracting that product from said total number of nucleotides in the reference nucleotide. Alterations of a polynucleotide sequence encoding the polypeptide may alter the polypeptide encoded by the polynucleotide following such alterations.

Similarly, a polypeptide sequence of the present disclosure may be identical to the reference sequence of SEQ ID NO: 2, that is be 100% identical, or it may include up to a certain integer number of amino acid alterations as compared to the reference sequence such that the % identity is less than 100%. Such alterations are selected from the group including of at least one amino acid deletion, substitution, including conservative and non-conservative substitution, or insertion, and wherein said alterations may occur at the amino- or carboxy-terninal positions of the reference polypeptide sequence or anywhere between those terminal positions, interspersed either individually among the amino acids in the reference sequence or in one or more contiguous groups within the reference sequence. The number of amino acid alterations for.a given % identity is determined by multiplying the total number of amino acids in the reference polypeptide by the numerical percent of the respective percent identity (divided by 100) and then subtracting that product from said total number of amino acids in the reference polypeptide.

The terms “amino-terminal” and “carboxyl-terrninal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (e.g., GAU and GAC triplets each encode Asp).

The term “expression vector” is used to denote a DNA molecule, linear or circular, which includes a segment encoding a polypeptide of interest operably linked to additional segments that provide for its transcription and translation. Such additional segments include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated polynucleotide molecules of the present disclosure are free of other polynucleotides with which they are ordinarily associated, but may include naturally occurring 5′ and 3′ untranslated regions such as promoters and terminators. The identification of associated regions will be evident to one of ordinary skill in the art (Dynan, et al., Nature, 316: 774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes (e.g., transcription initiates in the promoter and proceeds through the coding segment to the terminator).

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

The term “modulate” and “modulation” denote adjustment or regulation of the activity of a compound or the interaction between one or more compounds.

The term “phenotype” means a property of an organism that can be detected, which is usually produced by interaction of an organism's genotype and environment.

The term “open reading frame” means the amino acid sequence encoded between translation initiation and termination codons of a coding sequence.

The term “codon” means a specific triplet of mononucleotides in the DNA chain. Codons correspond to specific amino acids (as defined by the transfer RNAs) or to start and stop of translation by the ribosome.

The term “wild-type” means that the nucleic acid fragment does not include any deleterious mutations. A “wild-type” protein means that the protein is active at a level of activity found in nature and includes the amino acid sequence found in nature.

The term “chimeric protein” means that the protein comprises regions which are wild-type and regions which are mutated. It may also mean that the protein comprises wild-type regions from one protein and wild-type regions from another protein.

The term “mutation” means a change in the sequence of a wild-type nucleic acid sequence or a change in the sequence of a polypeptide. Such mutation may be a point mutation such as a transition or a transversion. The mutation may be a deletion, an insertion, a substitition or a duplication.

In the polypeptide notation used herein, the lefthand direction is the amino terminal direction and the righthand direction is the carboxy-terminal direction, in accordance with standard usage and convention. Similarly, unless specified otherwise, the lefthand end of single-stranded polynucleotide sequences is the 5′ end; the lefthand direction of double-stranded polynucleotide sequences contains the 5′ end of the top strand, and the 3′ end of the bottom strand.

The term “agent” is used herein to denote a chemical compound, a mixture of chemical compounds, an array of spatially localized compounds (e.g., a VLSIPS peptide array, polynucleotide array, and/or combinatorial small molecule array), a biological macromolecule, a bacteriophage peptide display library, a bacteriophage antibody (e.g., scFv) display library, a polysome peptide display library, or an extract made from biological materials such as bacteria, plants, fungi, or animal (particularly mammalian) cells or tissues.

All publications, including but not limited to patents and patent applications, cited in this specification are herein incorporated by reference as if each individual publication were specifically and individually indicated to be incorporated by reference herein as though fully set forth.

As indicated above, embodiments of the present disclosure include polypeptides and polynucleotides that encode the polypeptides. Embodiments of the polypeptide are designated “GALE polypeptides”, while embodiments of the polynucleotides are designated “GALE polynucleotides.” One GALE polynucleotide sequence is set forth in SEQ ID NO: 1 (C307YhGALE) and the corresponding GALE polypepetide amino acid sequence is set forth in SEQ ID NO: 2. A second GALE polynucleotide sequence is set forth in SEQ ID NO: 3 (WTeGALE) and the corresponding GALE polypeptide sequence is set forth in SEQ ID NO: 4.

As discussed above, embodiments of the present disclosure provide GALE polynucleotides, including DNA and RNA molecules that encode the GALE polypeptides. Those skilled in the art will readily recognize that, in view of the degeneracy of the genetic code, considerable sequence variation is possible among these polynucleotide molecules. SEQ ID NO: 1 and SEQ ID NO: 3 are degenerate polynucleotide sequences that encompass polynucleotides that encode the GALE polypeptides of SEQ ID NO: 2 and SEQ ID NO: 4. The degeneracy of nucleic acid is well known in the art and as such degenerate polynucleotides of SEQ ID NO: 1 and SEQ ID NO.3 are included within the scope of the present disclosure.

Table 1 sets forth the three letter symbols and the one letter symbols for the amino acids as well as possible codons that can be associated with the amino acids.

TABLE 1

| THREE | ONE LETTER | SYNONYMOUS |
|---|---|---|
| LETTER CODE | CODE | CODONS |
| Cys | C | TGC TGT |
| Ser | S | AGC AGT TCA TCC |
| | | TCG TCT |
| Thr | T | ACA ACC ACG ACT |
| Pro | P | CCA CCC CCG CCT |
| Ala | A | GCA GCC GCG GCT |
| Gly | G | GGA GGC GGG GGT |
| Asn | N | AAC AAT |
| Asp | D | GAC GAT |
| Glu | E | GAA GAG |
| Gln | Q | CAA CAG |
| His | H | CAC CAT |
| Arg | R | AGA AGG CGA CGC |
| | | CGG CGT |
| Lys | K | AAA AAG |
| Met | M | ATG |
| Ile | I | ATA ATC ATT |
| Leu | L | CTA CTC CTG CTT |
| | | TTA TTG |
| Val | V | GTA GTC GTG GTT |
| Phe | F | TTC TTT |
| Tyr | Y | TAC TAT |
| Trp | W | TGG |
| Asn-Asp | B | |
| Glu-Gln | Z | |
| Any | X | |

One of ordinary skill in the art will appreciate that some ambiguity is introduced in determining a degenerate codon. Other nucleic acid sequences that encode the same protein sequence are considered equivalents. Thus, some polynucleotides encompassed by the degenerate sequence may encode variant amino acid sequences, but one of ordinary skill in the art can easily identify such variant sequences by reference to the amino acid sequences of SEQ ID NO: 2 and SEQ ID NO: 4.

Variant GALE polynucleotides that encode polypeptides that can be used as defined above are within the scope of the embodiments of the present disclosure. More specifically, variant GALE polynucleotides that encode polypeptides which exhibit at least about 50%, about 75%, about 85%, and preferably about 90%, of the activity of GALE polypeptides encoded by the variant GALE polynucleotides are within the scope of the embodiments of the present disclosure.

For any GALE polypeptide, including variants and fusion proteins, one of ordinary skill in the art can readily generate a fully degenerate polynucleotide sequence encoding that variant using the information set forth in Table 1. Moreover, those of skill in the art can use standard software to devise GALE variants (i.e., polynucleotides and polypeptides) based upon the polynucleotide and amino acid sequences described herein.

As indicated above, GALE polynucleotides and isolated GALE polynucleotides of the present disclosure can include DNA and RNA molecules. Methods for preparing DNA and RNA are well known in the art. In general, RNA is isolated from a tissue or cell that produces GALE RNA. Such tissues and cells can be identified by Northern blotting (Thomas, Proc. Natl. Acad. Sci. USA, 77: 5201, 1980). An exemplary source being human liver tissue. Total RNA can be prepared using guanidine HCI extraction followed by isolation by centrifugation in a CsCl gradient (Chirgwin, et al., Biochemistry, 18:,52-94, 1979). Complementary DNA (CDNA) can be prepared from the RNA using known methods. In the alternative, genomic DNA can be isolated. Polynucleotides encoding GALE polypeptides are then identified and isolated by hybridization or PCR, for example.

GALE polynucleotides can also be synthesized using techniques widely known in the art. (Glick, et al., Molecular Biotechnology, Principles & Applications of Recombinant DNA, (ASM Press, Washington, D.C. 1994); Itakura, et al., Annu. Rev. Biochem., 53: 323-56, 1984 and Climie, et al., Proc. Natl. Acad. Sci. USA, 87: 633-7, 1990.

Embodiments of the present disclosure also provide for GALE polypeptides and isolated GALE polypeptides that are substantially homologous to the GALE polypeptides of SEQ ID NO: 2 and SEQ ID NO: 4. The term “substantially homologous” is used herein to denote polypeptides having about 50%, about 75%, about 85%, and preferably about 90% sequence identity to the sequence shown in SEQ ID NO: 2 and SEQ ID NO: 3. Percent sequence identity is determined by conventional methods as discussed above. In addition, embodiments of the present disclosure include polynucleotides that encode homologous polypeptides.

In general, homologous polypeptides are characterized as having one or more amino acid substitutions, deletions, and/or additions. These changes are preferably of a minor nature, that is conservative amino acid substitutions and other substitutions that do not significantly affect the activity of the polypeptide; small substitutions, typically of one to about six amino acids; and small amino- or carboxyl-terminal extensions, such as an amino-terminal methionine residue, a small linker peptide of up to about 2-6 residues, or an affinity tag. Homologous polypeptides comprising affinity tags can further comprise a proteolytic cleavage site between the homologous polypeptide and the affinity tag.

In addition, embodiments of the present disclosure include polynucleotides that encode polypeptides having one or more “conservative amino acid substitutions,“compared with the GALE polypeptides of SEQ ID NO: 2 and SEQ ID NO: 4. Conservative amino acid substitutions can be based upon the chemical properties of the amino acids. That is, variants can be obtained that contain one or more amino acid substitutions of SEQ ID NO: 2 and SEQ ID NO: 4, in which an alkyl amino acid is substituted for an alkyl amino acid in a GALE polypeptide, an aromatic amino acid is substituted for an aromatic amino acid in a GALE polypeptide, a sulfur-containing amino acid is substituted for a sulfur-containing amino acid in a GALE polypeptide, a hydroxy-containing amino acid is substituted for a hydroxy-containing amino acid in a GALE polypeptide, an acidic amino acid is substituted for an acidic amino acid in a GALE polypeptide, a basic amino acid is substituted for a basic amino acid in a GALE polypeptide, or a dibasic monocarboxylic amino acid is substituted for a dibasic monocarboxylic amino acid in a GALE polypeptide.

Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. Other conservative amino acid substitutions are provided in Table 2.

TABLE 2

| CHARACTETISTIC | AMINO ACID |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Conservative amino acid changes in GALE polypeptides can be introduced by substituting nucleotides for the nucleotides recited in SEQ ID NO: 1 and SEQ ID NO: 3. Such “conservative amino acid” variants can be obtained, for example, by oligonucleotide-directed mutagenesis, linker-scanning mutagenesis, mutagenesis using the polymerase chain reaction, and the like (McPherson (Ed.), Directed Mutagenesis: A Practical Approach (IRL Press 1991)). The ability of such variants to treat conditions as well as other properties of the wild-type protein can be determined using standard methods. Alternatively, variant GALE polypeptides can be identified by the ability to bind specifically to anti-GALE antibodies.

GALE polypeptides having conservative amino acid variants can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methyl-glycine, allo-threonine, methylthreonine, hydroxy-ethylcysteine, hydroxyethylhomocysteine, nitro-glutamine, homoglutamine, pipecolic acid, thiazolidine carboxylic acid, dehydroproline, 3- and 4-methylproline, 3,3-dimethylproline, tert-leucine, norvaline, 2-azaphenyl-alanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. (Robertson, et al., J. Am. Chem. Soc., 113: 2722, 1991; Ellman, et al., Methods Enzymol., 202: 301, 1991; Chung, et al., Science, 259: 806-9, 1993; and Chung, et al., Proc. Natl. Acad. Sci. USA, 90: 10145-9, 1993). In a second method, translation is carried out in Xenopus oocytes by microinjection of mutated mRNA and chemically aminoacylated suppressor tRNAs (Turcatti, et al., J. Biol. Chem., 271: 19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. (Koide, et al., Biochem., 33: 7470-6, 1994). Naturally occurring amino acid residues can be converted to non-naturally occurring species by in vitro chemical modification. Chemical modification can be combined with site-directed mutagenesis to further expand the range of substitutions (Wynn, et al., Protein Sci., 2: 395-403, 1993).

Essential amino acids in the polypeptides of the present disclosure can be identified according to procedures known in the art, such as site-directed mutagenesis or alanine-scanning mutagenesis (Cunningham, et al., Science, 244: 1081-5, 1989; Bass, et al., Proc. Natl. Acad. Sci. USA, 88: 4498-502, 1991). In the latter technique, single alanine mutations are introduced at every residue in the molecule, and the resultant mutant molecules are tested for biological activity as disclosed below to identify amino acid residues that are critical to the activity of the molecule. (Hilton, et al., J. Biol. Chem., 271: 4699-708, 1996). Sites of ligand-receptor interaction can also be determined by physical analysis of structure, as determined by such techniques as nuclear magnetic resonance, crystallography, electron diffraction or photoaffinity labeling, in conjunction with mutation of putative contact site amino acids. (de Vos, et al., Science, 255: 306-12, 1992; Smith, et al., J. Mol. Biol., 224: 899-904, 1992; Wlodaver, et al., FEBS Lett., 309: 59-64, 1992). The identities of essential amino acids can also be inferred from analysis of homologies with related nuclear membrane bound proteins.

Multiple amino acid substitutions can be made and tested using known methods of mutagenesis and screening, such as those disclosed by Reidhaar-Olson and Sauer (Science, 241: 53-7, 1988) or Bowie and Sauer (Proc. Natl. Acad. Sci. USA, 86: 2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (Lowman, et al., Biochem., 30: 10832-7, 1991; Ladner, et al., U.S. Pat. No. 5,223,409) and region-directed mutagenesis (Derbyshire, et al., Gene, 46:145, 1986; Ner, et al., DNA, 7:127, 1988).

Variants of the disclosed GALE polypeptides can be generated through DNA shuffling. (Stemmer, Nature, 370: 389-91, 1994 and Stemmer, Proc. Natl. Acad. Sci. USA, 91: 10747-51, 1994). Briefly, variant polypeptides are generated by in vitro homologous recombination by random fragmentation of a parent DNA followed by reassembly using PCR, resulting in randomly introduced point mutations. This technique can be modified by using a family of parent DNAs, such as allelic variants or genes from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods can be combined with high-throughput, automated screening methods to detect activity of cloned, mutagenized polypeptides in host cells. Preferred assays in this regard include cell proliferation assays and biosensor-based ligand-binding assays. Mutagenized DNA molecules that encode active polypeptides can be recovered from the host cells and rapidly sequenced using modern equipment. These methods allow the rapid determination of the importance of individual amino acid residues in a polypeptide of interest, and can be applied to polypeptides of unknown structure.

Using the methods discussed herein, one of ordinary skill in the art can identify and/or prepare a variety of GALE polypeptide fragments or variants of SEQ ID NO: 2 of SEQ ID NO: 4 that retain the functional properties of the GALE polypeptides. Such polypeptides may also include additional polypeptide segments as generally disclosed herein.

For any GALE polypeptide, including variants and fusion proteins, one of ordinary skill in the art can readily generate a degenerate polynucleotide sequence encoding that variant using the information set forth in Table 1 above as well as what is known in the art.

As used herein, a fusion protein consists essentially of a first portion and a second portion joined by a peptide bond. In one embodiment the first portion includes a polypeptide comprising a sequence of amino acid residues that is at least about 50%, about 75%, about 85%, and preferably about 90% identical in amino acid sequence to SEQ ID NO: 2 or SEQ ID NO: 4 and the second portion is any other heterologous non GALE polypeptide. The other polypeptide may be one that does not inhibit the function of the GALE polypeptide, such as a signal peptide to facilitate secretion of the fusion protein or an affinity tag.

The GALE polypeptides of the present disclosure, including full-length polypeptides, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells. (Sambrook et al., Molecular Cloning: A Laboratory Manual, 2nd Ed., Cold Spring Harbor Laboratory Press, Cold Spring Harbor, N.Y., 1989, and Ausubel, et al., Eds., Current Protocols in Molecular Biology, John Wiley and Sons, Inc., N.Y., 1987).

In general, GALE polynucleotide sequences encoding GALE polypeptides are operably linked to other genetic elements required for its expression, generally including a transcription promoter and terminator, within an expression vector. The vector also commonly contains one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

It is preferred to purify the GALE polypeptides of the present disclosure to about 80% purity, more preferably to about 90% purity, even more preferably about 95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant GALE polypeptides (or fusion GALE polypeptides) can be purified using fractionation and/or conventional purification methods and media. Ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. (Affinity Chromatography: Principles & Methods, Pharmacia LKB Biotechnology, Uppsala, Sweden, 1988).

The GALE polypeptides of the present disclosure can be isolated by exploitation of their binding properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem., 3: 1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., 182, M. Deutscher, (Ed.), Acad. Press, San Diego, 1990, pp.529-39). Within additional embodiments of the disclosure, a fusion of the polypeptide of interest and an affinity tag (e.g., Gly-Gly tag) may be constructed to facilitate purification.

GALE polypeptides or fragments thereof may also be prepared through chemical synthesis according to methods known in the art, including exclusive solid phase synthesis, partial solid phase methods, fragment condensation or classical solution synthesis. (Merrifield, J. Am. Chem. Soc., 85: 2149, 1963).

Using methods known in the art, GALE polypeptides may be prepared as monomers or multimers and may be post-translationally modified or unmodified.

### EXAMPLE 1

Preparation and Expression of SEQ ID NO: 1 (C307y h GALE): Site-directed PCR mutagenesis was performed on the WThGALE cDNA sequence using the following primers: SEQ ID NO: 5-hEPIMFC307Y, 5′-GGTGATGTGGCAGCCTATTACGCCAACCCC-3′ and SEQ ID NO: 6-hEPIMRC307Y, 5′-GCTGGGGTTGGCGTAATAGGCTGCCACATCACC-3′. Following mutagenesis, dideoxy sequencing was performed to confirm mutation and remaining wild-type sequence. The mutations of interest were introduced into the high copy number Pichia pastoris expression vector pPIC3.5K (Invitrogen), which already contained WThGALE sequence, by gap repair in the bacterial strain XL-1 blue, and again confirmed by sequencing. It will be appreciated that other host cells and expression vectors may be utilized. Plasmids were then introduced into the methylotrophic yeast, Pichia pastoris for protein overexpression. Plasmids were linearized and integrated in multiple copy into the Pichia strain, GS115, using a spheroplasting kit (Invitrogen). Cells were screened and selected on G418 (U.S. Biological) for the highest expressing colonies. Expression was confirmed by western blot analysis as previously described in Wohlers et al. Am. J. Hum. Gen. 64:462-470(1999). Clones demonstrating the highest level of hGALE expression were then expanded, cultured, and induced for expression with methanol in a New Brunswick Scientific Bioflo 3000 fermenter. Cells were lysed by agitation with glass beads in breaking buffer (50 mM sodium phosphate pH 7.4, 1 mM PMSF, 1 mM EDTA and 5% glycerol) using a Beadbeater (Biospec). Cell lysates were collected and the soluble portion retrieved by centrifuging spinning at 4° C. in a high-speed centrifuge (Sorvall) until the supernatant was clear. The wild-type and C307Y mutant epimerases were purified and crystallized precisely as previously described (Thoden, 1996).

### EXAMPLE2

In Vitro Assays for UDP-Gal: Aliquots of each purified enzyme from Example lwere stored in 50% glycerol with 4 mM NAD+ in liquid nitrogen, while crude extracts were stored at −80° C. until needed. All crude extracts were passed through Micro biospin 30 columns (Biorad) before being assayed for enzyme activity. Assays to determine the level of GALE activity with respect to UDP-Gal were performed essentially as previously described in Thoden et al. J Biol Chem Jul 26; 277(30):27528-34 (2002). Enzymatic conversion from substrate to product was detected either by radioactive assay or by carbohydrate analysis on HPLC; results from the HPLC assays were determined to be comparable to those seen for the radioactive assay (data not shown). For radioactive assay, conversion of UDP-Gal to UDP-Glc was measured in a 12.5-μl reaction containing 2.5 μl of premix (0.05 μCi of UDP-[¹⁴C]Gal (Amersham Biosciences), 2 nM cold UDP-Gal, 0.2 mM glycine buffer, pH 8.7), 2.5 μl of 20 mM NAD+, and 7.5 μl of purified protein diluted in Johnston buffer (20 mM HEPES/KOH, pH 7.5, 1 mM dithiothreitol, and 0.3 mg of bovine serum albumin/ml). Appropriate amounts of protein were used in each reaction in order to stay within the predetermined linear range of the assay. Reactions were incubated at 37° C for 30 min and were stopped by boiling at 100° C. for 10 min. Following high speed centrifugation for 15 min in a microcentrifuge, 10 μl of the sample was spotted onto a prewashed PEI-Cellulose TLC plate (Baker). After thorough drying, the plate was run for 16-24 h in a solvent containing 1.5 mM Na₂B₄O₇, 5 mM H₃BO₃, and 25% ethylene glycol. After running, plates were air-dried before being exposed to storage phosphor screens (Amersham Biosciences) overnight. Images were visualized with a Typhoon 9200 variable mode imager and quantified using ImageQuant software (both from Amersham Biosciences). Percent conversion was determined by dividing the product signal by the total signal and multiplying by 100. For detection by HPLC, the above assay protocol was used, with minor modifications. C 14-labeled UDP-galactose was removed from the premix, and the corresponding volume replaced by water. The assay proceeded through the 30 min incubation described above, and was then stopped by addition of 2.5 volumes of ice cold 100% methanol. After brief vortex mixing, samples were spun on high speed for 10 min at 4° C. Supernatant was collected, and dried under vacuum with low heat. Resultant pellets were resuspended in 250μl ddH₂O, and the suspension added to an 0.2 μM nylon micro-spin filter tube (Alltech), and spun for approximately 5 min at 4000g. A 15 μl aliquot was then analyzed by HPLC.

In Vitro Assay for UDP-GalNAc: The radioactive method for detecting conversion of UDP-GalNAc to UDP-GIcNAc was performed essentially as described above for UDP-Gal, with the following assay components per 25 μl of reaction: 8.75 μl of premix (0.04 μCi of UDP-[¹⁴C]GalNAc (ICN), 1.89 mM cold UDP-GalNAc, 28.6 mM pyruvate, 286 mM glycine, pH 8.7, 5 μl of 20 mM NAD), and 11.25 μl of protein diluted in Johnston buffer. Appropriate amounts of protein were used in each reaction to stay within the predetermined linear range of the assay. Assays were performed as for UDP-Gal, with a TLC run-time of 10 h and quantified as described for UDP-Gal.

For analysis by HPLC, protein samples were diluted with glycine buffer (100 mM glycine, pH 8.7) to a final volume of 7.5 μl. For each reaction, 2.5 μl of 20 mM NAD+, and 2.5 il of premix (3.3 mM UDP-GalNAc, and 500 mM glycine, pH 8.7) were added, for a final reaction volume of 12.5 μl. Assay mixtures were incubated at 37° C. for 30 min before stopping by addition of 2.5 volumes of ice-cold 100% methanol. Samples were vortexed, spun and dried as for UDP-Gal HPLC assays, and resuspended in 750 μl ddH₂O. The suspension was added to an 0.2 μm nylon micro-spin filter tube, and spun for approximately 2.5 min at 4000 g. An aliquot of 20 μl was then analyzed by HPLC.

HPLC Analysis of Carbohydrates: Carbohydrate detection by HPLC was based on the methods of Smits (1998) and de Koning (1992). HPLC analysis was carried out on a DX600 HPLC system (Dionex, Sunnyvale, CA) consisting of a Dionex AS50 autosampler, a Dionex GP50 gradient pump, and a Dionex ED50 electrochemical detector. Carbohydrates were separated on a CarboPac PA10 column, 250×4 mm, with a CarboPac PA10 guard column, 50×4 mm, placed before the analysis column, and a borate trap placed after. It was noted that elimination of the borate trap led to better separation of UDP-sugars from NAD; therefore, the trap was removed for all UDP-GalNAc analyses. For UDP-Gal assays 15 μl was injected into a 25 μl injection loop, while for UDP-GalNAc assays, the injection volume was 20 μl. Samples were maintained at 4° C. in the autosampler tray and the HPLC analysis was carried out at room temperature.

The following mobile phase buffers were used for HPLC analysis: buffer A, 15 mM NaOH, and buffer B, 50 mM NaOH/1 M NaAC. To prevent carbonate contamination of the analysis column, a 50% NaOH solution (Fisher) containing less than 0.04% sodium carbonate was used. Buffers were degassed with He and then maintained under an He atmosphere. UDP-Gal and UDP-Glc were separated using a high salt isocratic procedure with a flow rate of 1 mmin: 30% buffer A and 70% buffer B for 20 min. UDP-GalNAc and UDP-GlcNAc were separated using an isocratic procedure with a flow rate of 0.75 ml/min: 45% buffer A and 55% buffer B for 40 min.

The ED50 detector consisted of a gold electrode and a pH-Ag/AgCl reference electrode for signal detection by integrated amperometry. The following waveform potential-time sequence was used: 0.1 V (0 to 0.20 s), with integration at 0.1 V (0.20 to 0.40 s), followed by a decrease to −2.0 V (0.41 to 0.42 s), increase to 0.6 V (0.43 s), decrease to −0.10 V (0.44 to 0.50 s). Carbohydrates were quantified using PeakNet software version 6.4 (Dionex) and based on integration of peak areas with comparison to standards. For evaluation of UDP-hexoses, the following standard solution (1×) was used: 10 μM UDP-GalNAc, 10 μM UDP-GlcNAc, 100 μM UDP-Gal, and 100 μM UDP-Glc.

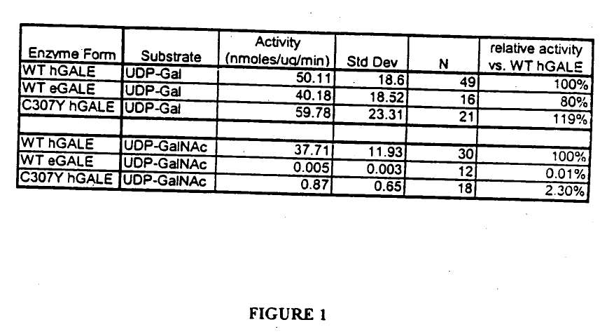

As shown in FIG. 1, the in vitro activity assays were performed to determine the ability of each purified enzyme, wild type human GALE (WThGALE), wild-type E. coli GALE (WTeGALE) and the mutant human enzyme C307YhGALE, to epimerize the substrates, UDP-gal and UDP-galNAc. These recombinant proteins were all expressed in and purified from Pichia Pastoris. As demonstrated, WT eGALE has no ability to interconvert UDP-GalNAc and UDP-GlcNAc, while WT hGALE can interconvert both UDP-Gal /UDP-Glc, and UDP-GalNAc /UDP-GlcNAc well. The C307Y hGALE protein maintains wildtype levels of UDP-Gal activity, while UDP-GalNAc activity is reduced to 2.30% of that seen in WT hGALE.

### EXAMPLE 3

Construction of Vectors:

GALE vectors: All GALE alleles were introduced into the CMV promoter-driven mammalian expression vector, pCDNA3 (Invitrogen), which contains a G418 resistance gene for selection of stable cell lines. The allele sequences contained a HA affinity tag for monitoring the stable expression of the GALE protein in cells. In order to obtain a level of GALE expression, which is comparable to endogenous levels seen in CHO-KI cells, it was necessary to remove the CMV promoter in some vectors, and replace it with the weaker mouse Galactose-1-Phosphate Uridylyltransferase (mGALT) promoter. The mGALT promoter sequence was obtained by PCR-amplification of the promoter sequence from crude mouse genomic DNA. The primers used to create the mGALT sequence contained the restriction enzyme sequences Mlu I and Hind III for ease of sub-cloning: mGALTproMlulfl, 5′-CGCGACGCGTATCCGTGGCGGGACGAATGGACACAGCAAC-3′ (SEQ ID NO: 7) and mGALTproHind3rl, 5′-CGCGAAGCTTATCGGCTCCGCTATGCGACGTGAGGCC-3′ (SEQ NO: 8). The PCR product was subcloned into the pCDNA3 vector, replacing the CMV promoter, and finally subjected to dideoxy sequencing to ensure correct sequence.

### EXAMPLE 4

Transfection and isolation ofstable clones containing SEQ ID NO: 1 (C307Y h GALE): ldlD cells were transfected with the mammalian expression vector, pCDNA3 (Invitrogen), encoding an HA-tagged allele of C307Y hGALE, and subcloned by standard recombinant techniques and using standard protocols for the lipofection reagents Lipofectamine 2000 or Lipofectamine (both by Invitrogen). Cells were re-plated at <1:10 in selective media containing G418 (U.S. Biologicals). After approximately 14d of drug selection, individual clones were isolated and purified by further exposure to selective drugs. Stable expression of GALE alleles in said clones was confirmed by western blot analysis targeting the HA-tag, and by activity assays.

Cell culture methods: ldlD cells, and the parent cell line, CHO-KI were maintained under standard protocols (trypsin-EDTA harvesting) and conditions (5% CO₂, 37° C.) in a monolayer culture in Ham's F-12 media (containing 100 U/ml Penicillin, 100 pg/ml streptomycin, 2 mM glutamine, and 5% (v/v) fetal bovine serum (FBS)). For experiments, cells were EDTA-trypsin harvested, and washed with media before being counted and plated at the appropriate densities. In experiments studying glycosylation or galactose sensitivity, it is necessary to avoid the use of serum containing large amounts of glycoproteins from which Gal and GalNAc can be scavenged (Krieger, 1989). For this reason, 5% FBS in these experiments must be replaced by one of the following: (i) direct plating into 1-3% NCLPDS; (ii) plating into 1-3% NCLPDS for Id, followed by the replacement of this media with ITS+ medium (0.625 mg/ml insulin, 0.625 mg/ml transferring, 0.625 ug/ml selenium, 0.535 mg/ml linoleic acid, and 0.125 g/ml BSA), or an equivalent culture medium containing less glycoproteins/glycolipids than 5% FBS to allow expression of the phenotype (Krieger et al. 1986).

Preparation of lipoprotein-deficient serum: Newborn calf lipoprotein-deficient serum (NCLPDS) was made according to the method described by Goldstein, and modified by Krieger et al.(1986). Whole newborn calf serum (Invitrogen) was adjusted to a final density of 1.215 g/ml with solid Potassium Bromide (Sigma). The serum was then centrifuged for 36 hr at 4° C. and 59,000 RPM in a 60 Ti Beckman rotor. The resulting bottom layer (deficient in lipoproteins) was separated from the lipoprotein-containing fraction. The lipoprotein-deficient fraction was dialyzed at 4° C. against a total of 30 L of 150 mM NaCl for 72hr, changing dialyzing liquid 5 times. The lipoprotein-deficient serum was sterilized with a 0.45 μM Millipore filter and adjusted to a protein concentration of 60 mg/ml by dilution with 150 mM NaCl. This procedure results in a total serum cholesterol content, which is <5% of that found in the initial whole serum.

Western Blot Analyses: Western blot analyses were performed as described previously(Lang, Li, Black-Brewster, and Fridovich-Keil, Nucleic Acids Research 29: 2567-2574 (2001). HA-tagged GALE protein alleles were detected using the 12CA5 monoclonal antibody (mAb, Roche) at a final concentration of 0.8 μg/ml followed by HRP-conjugated donkey anti-mouse secondary antibody (Covance), diluted 1:5000. Signals were detected by chemiluminescence. Immediately before incubation, 1.5μl of 30% (w/w) H₂O₂ were added to 10 ml of a working solution (1.25 mM luminol, 0.2 mM p-coumaric acid, and 100 mM Tris-HCL, pH 8.5). The resultant solution was added to the nitrocellulose blot, and incubated for 2 minutes before exposure to film.

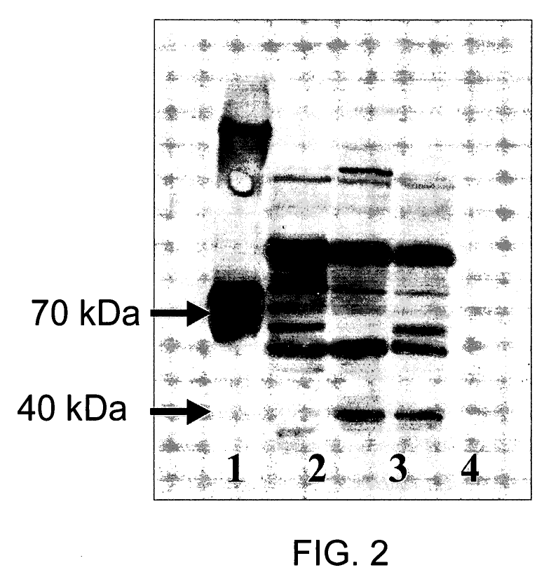

It has been demonstrated that IdiD cells transfected with C307YhGALE do express C307YhGALE. Protein extracts from ldlD cells, IdiD stably expressing WThGALE, and ldlD stably expressing C307YGALE were subjected to SDS-PAGE, and analyzed by western blot. Both the C307YhGALE and hGALE proteins contained an HA tag. The results, demonstrating expression of both 40 kDa epimerase proteins, are shown in FIG. 2. Each lane contains 50 ug protein. GALE enzyme is represented by a band at 40 kDa. Lane 1, marker; lane 2, IdiD cells; lane 3, positive control (ldlD cells transfected w/HA-tagged WT human GALE); lane 4, ldlD cells transfected with C307Y human GALE.

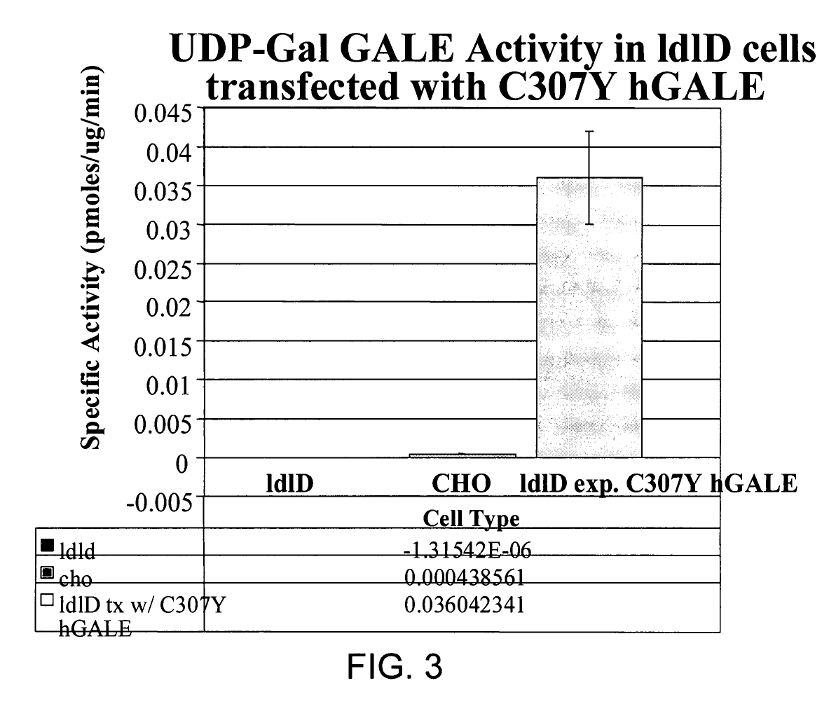

It was further demonstrated that the C307YhGALE expressed in ldlD cells is active. Protein extracts from ldlD cells, CHO cells and ldlD cells stably expressing C307YhGALE driven by the CMV promoter were subjected to in vitro UDP-gal activity assays. CHO cells were used as a positive control and ldlD cells were used as a negative control. The results are shown in FIG. 3.

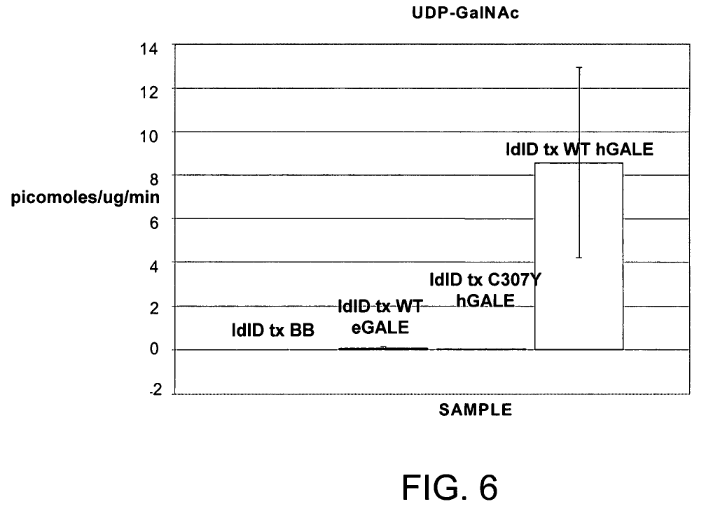

Finally, while C307YhGALE expressed in ldlD cells is active with respect to UDP-Gal, the activity with respect to UDP-GalNAc is reduced to levels close to those seen in ldld cells expressing backbone alone, as demonstrated in FIG. 6. In this experiment, ldlD cells expressing WThGALE were used as a positive control, and ldlD cells expressing backbone alone were used as a negative control. Without the ability to produce UDP-GalNAc endogenously from UDP-GlcNAc, these cells will be dramatically reduced in their capacity to synthesize 0-glycans without the addition of exogenous sugars, while the ability to synthesize N-glycans will be maintained.

### EXAMPLE 5

Transfection and isolation of stable clones containing SEQ ID NO: 3 (WTeGALE): ldlD cells were transfected with the mammalian expression vector, pCDNA3 (Invitrogen), encoding an HA-tagged allele of otherwise WTeGALE, which had been amplified from E. coli genomic DNA, and subcloned by standard recombinant techniques and using standard protocols for the lipofection reagents Lipofectamine 2000 or Lipofectamine (both by Invitrogen). Cells were re-plated at <1:10 in selective media containing G418 (U.S. Biologicals). After approximately 14d of drug selection, individual clones were isolated and purified by further exposure to selective drugs. Stable expression of GALE alleles in said clones was confirmed by western blot analysis targeting the HA-tag, and by activity assays.

Cell culture methods: As described in Example 4 above.

Preparation of lipoprotein-deficient serum: As described in Example 4 above.

Western Blot Analyses: Western blot analyses were performed as described previously (Lang, Li, Black-Brewster, and Fridovich-Keil, Nucleic Acids Research 29: 2567-2574 (2001). HA-tagged GALE protein alleles are detected using the 12CA5 monoclonal antibody (mAb, Roche) at a final concentration of 0.8 μg/ml followed by HRP-conjugated donkey anti-mouse secondary antibody (Covance), diluted 1:5000. Signals were detected by chemiluminescence. Immediately before incubation, 1.5μl of 30% (w/w) H₂O₂ were added to 10 ml of a working solution (1.25 mM luminol, 0.2 mM p-coumaric acid, and 100 mM Tris-HCL, pH 8.5). The resultant solution was added to the nitrocellulose blot, and incubated for 2 minutes before exposure to film.

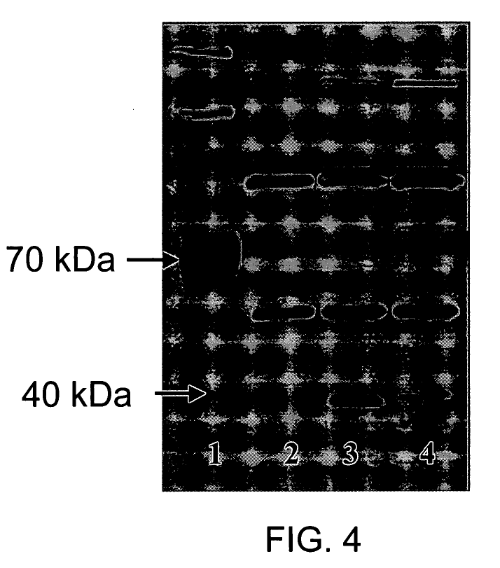

It has been demonstrated that ldlD cells transfected with WTeGALE do express WTeGALE. Protein extracts from ldlD cells, ldlD stably expressing WThGALE, and ldlD stably expressing WTeGALE were subjected to SDS-PAGE, and analyzed by western blot. Both the eGALE and hGALE proteins contained an HA tag. The results are shown in FIG. 4. Each lane contains 50 ug protein. GALE enzyme is represented by a band at 4OkDa. Lane 1, marker; lane 2, ldlD cells; lane 3, positive control (ldlD cells transfected w/HA-tagged WT human GALE); lane 4, ldlD cells transfected with WT E. coli GALE.

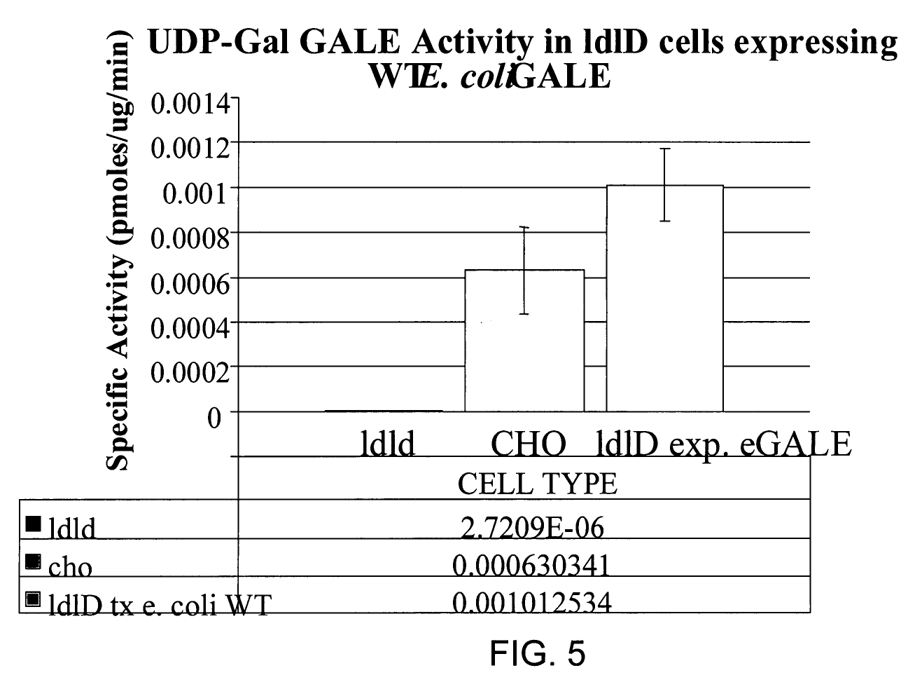

It was further demonstrated that the WTeGALE expressed in ldlD cells is active. Protein extracts from ldlD cells, or ldlD cells stably expressing WTeGALE driven by the CMV promoter were subjected to in vitro UDP-gal activity assays. CHO cells were used as a positive control and ldlD cells were used as a negative control. The results are shown in FIG. 5.

Finally, while C307YhGALE expressed in ldlD cells is active with respect to UDP-Gal, the activity with respect to UDP-GalNAc is reduced to levels close to those seen in IdiD cells expressing backbone alone, as demonstrated in FIG. 6. In this experiment, ldlD cells expressing WThGALE were used as a positive control, and ldlD cells expressing backbone alone were used as a negative control. Without the ability to produce UDP-GalNAc endogenously from UDP-GlcNAc, these cells will be dramatically reduced in their capacity to synthesize O-glycans without the addition of exogenous sugars, while the ability to synthesize N-glycans will be maintained.

It should be emphasized that the above-described embodiments of the present disclosure are merely possible examples of implementations, and are merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

1. An isolated polynucleotide comprising a polynucleotide selected from: a polynucleotide sequence set forth in SEQ ID NO: 1 (C307YhGALE) or a degenerate variant of the SEQ ID NO: 1; a polynucleotide sequence at least 90% identical to the polynucleotide sequence set forth in SEQ ID NO: 1; a polynucleotide sequence at least 75% identical to the polynucleotide sequence set forth in SEQ ID NO: 1; and a polynucleotide sequence at least 50% identical to the polynucleotide sequence set forth in SEQ ID NO: 1.

2. A polypeptide selected from: an amino acid sequence set forth in SEQ ID NO: 2 (C307YhGALE), or conservatively modified variants thereof; an amino acid sequence that is at least 90% identical to SEQ ID NO: 2; an amino acid sequence that is at least 75% identical to SEQ ID NO: 2; and an amino acid sequence that is at least 50% identical to SEQ ID NO: 2.

3. A vector comprising the isolated polynucleotide of claim 1.

4. The vector of claim 3 wherein the vector is pPIC3.5K .

5. An isolated host cell comprising the vector of claim 3.

6. The isolated host cell of claim 5 wherein the host cell is selected from: Pichia pastoris, Saccharomyces cerevisiae, Schizosaccharomyces pombe, and Escherichia coli.

7. The isolated host cell of claim 6 wherein the host cell is Pichia pastoris.

8. A process for producing a polypeptide comprising culturing the host cell of claim 7 under conditions sufficient for the production of the polypeptide where the polypeptide has the characteristics that the polypeptide is capable of UDP-gal[UDP-glc interconversion and substantially incapable of UDP-galNAc[UDP-glcNAc interconversion.

9. The process of claim 8 wherein the polypeptide is the polypeptide of claim 2.

10. A cell line transfected with an expression vector comprising a polynucleotide selected from: a polynucleotide sequence set forth in SEQ ID NO: 1 (C307YhGALE) or a degenerate variant of the SEQ ID NO: 1; a polynucleotide sequence at least 90% identical to the polynucleotide sequence set forth in SEQ ID NO: 1; a polynucleotide sequence at least 75% identical to the polynucleotide sequence set forth in SEQ ID NO: 1; and a polynucleotide sequence at least 50% identical to the polynucleotide sequence set forth in SEQ ID NO: 1, encoding a polypeptide having the characteristics that the polypeptide is capable of UDP-gal/UDP-glc interconversion and substantially incapable of UDP-galNAc/UDP-glcNAc interconversion.

11. The cell line of claim 10 wherein the polypeptide is selected from: an amino acid sequence set forth in SEQ ID NO: 2 (C307YhGALE), or conservatively modified variants thereof; an amino acid sequence that is at least 90% identical to SEQ ID NO: 2; an amino acid sequence that is at least 75% identical to SEQ ID NO: 2; and an amino acid sequence that is at least 50% identical to SEQ ID NO: 2.

12. The cell line of claim 10 wherein the expression vector is pCDNA3.

13. The cell line of claim 10 wherein the cell line is GALE deficient.

14. The cell line of claim 13 wherein the cell line is ldlD.

15. A vector comprising an isolated polynucleotide selected from: a polynucleotide sequence set forth in SEQ ID NO: 3 (WTeGALE), or a degenerate variant of the SEQ ID NO: 3; a polynucleotide sequence at least 90% identical to the polynucleotide sequence set forth in SEQ ID NO: 3; a polynucleotide sequence at least 75% identical to the polynucleotide sequence set forth in SEQ ID NO: 3; and a polynucleotide sequence at least 50% identical to the polynucleotide sequence set forth in SEQ ID NO: 3.

16. The vector of claim 15 wherein the vector is pPIC3.5K.

17. An isolated host cell comprising the vector of claim 15.

18. The isolated host cell of claim 17 wherein the host cell is selected from: Pichia pastoris, Saccharomyces cerevisiae, Schizosaccharomyces pombe, and Escherichia coli.

19. The isolated host cell of claim 18 wherein the host cell is Pichiapastoris.

20. A process for producing a polypeptide comprising culturing the host cell of claim 19 under conditions sufficient for the production of the polypeptide where the polypeptide has the characteristics that the polypeptide is capable of UDP-gal/UDP-glc interconversion and substantially incapable of UDP-galNAc/UDP-glcNAc interconversion.

21. The process of claim 20 wherein the polypeptide is selected from: an amino acid sequence set forth in SEQ ID NO: 4, or conservatively modified variants thereof; an amino acid sequence that is at least 90% identical to SEQ ID NO: 4; an amino acid sequence that is at least 75% identical to SEQ ID NO: 4; and an amino acid sequence that is at least 50% identical to SEQ ID NO: 4

22. A cell line transfected with an expression vector comprising a polynucleotide selected from: a polynucleotide SEQ ID NO: 3 (WTeGALE) or a degenerate variant of the SEQ ID NO: 3; a polynucleotide sequence at least 90% identical to the polynucleotide sequence set forth in SEQ ID NO: 3; a polynucleotide sequence at least 75% identical to the polynucleotide sequence set forth in SEQ ID NO: 3; and a polynucleotide sequence at least 50% identical to the polynucleotide sequence set forth in SEQ ID NO: 3 encoding a polypeptide having the characteristics that the polypeptide is capable of UDP-gal/UDP-glc interconversion and substantially incapable of UDP-galNAc/UDP-glcNAc interconversion.

23. The cell line of claim 22 wherein the polypeptide is selected from: an amino acid sequence set forth in SEQ ID NO: 4 (WTeGALE), or conservatively modified variants thereof; an amino acid sequence that is at least 90% identical to SEQ ID NO: 4; an amino acid sequence that is at least 75% identical to SEQ ID NO: 4; and an amino acid sequence that is at least 50% identical to SEQ ID NO: 4

24. The cell line of claim 22 wherein the expression vector is pCDNA3.

25. The cell line of claim 22 wherein the cell line is GALE deficient.

26. The cell line of claim 25 wherein the cell line is ldlD.

27. A method of culturing the cell line of claim 10 in the absence of galactose to produce glycoproteins having N-linked modifications with substantially no O-linked modifications.

28. A method of culturing the cell line of claim 22 in the absence of galactose to produce glycoproteins having N-linked modifications with substantially no O-linked modifications.


SEQUENCE LISTING (8 sequences)

SEQ ID NO: 1 - DNA, 1047 bp, Homo Sapiens
atggcagagaaggtgctggtaacaggtggggctggctacattggcagccacacggtgctggagctgctggaggctggctacttgcctgtggtcatcgataacttccataatgccttccgtggagggggctccctgcctgagagcctgcggcgggtccaggagctgacaggccgctctgtggagtttgaggagatggacattttggaccagggagccctacagcgtctcttcaaaaagtacagctttatggcggtcatccactttgcggggctcaaggccgtgggcgagtcggtgcagaagcctctggattattacagagttaacctgaccgggaccatccagcttctggagatcatgaaggcccacggggtgaagaacctggtgttcagcagctcagccactgtgtacgggaacccccagtacctgccccttgatgaggcccaccccacgggtggttgtaccaacccttacggcaagtccaagttcttcatcgaggaaatgatccgggacctgtgccaggcagacaagacttggaacgcagtgctgctgcgctatttcaaccccacaggtgcccatgcctctggctgcattggtgaggatccccagggcatacccaacaacctcatgccttatgtctcccaggtggcgatcgggcgacgggaggccctgaatgtctttggcaatgactatgacacagaggatggcacaggtgtccgggattacatccatgtcgtggatctggccaagggccacattgcagccttaaggaagctgaaagaacagtgtggctgccggatctacaacctgggcacgggcacaggctattcagtgctgcagatggtccaggctatggagaaggcctctgggaagaagatcccgtacaaggtggtggcacggcgggaaggtgatgtggcagcctattacgccaaccccagcctggcccaagaggagctggggtggacagcagccttagggctggacaggatgtgtgaggatctctggcgctggcagaagcagaatccttcaggctttggcacgcaagcctga

SEQ ID NO: 2 - protein, 348 aa, Human polypeptide
MAEKVLVTGGAGYIGSHTVLELLEAGYLPVVIDNFHNAFRGGGSLPESLRRVQELTGRSVEFEEMDILDQGALQRLFKKYSFMAVIHFAGLKAVGESVQKPLDYYRVNLTGTIQLLEIMKAHGVKNLVFSSSATVYGNPQYLPLDEAHPTGGCTNPYGKSKFFIEEMIRDLCQADKTWNAVLLRYFNPTGAHASGCIGEDPQGIPNNLMPYVSQVAIGRREALNVFGNDYDTEDGTGVRDYIHVVDLAKGHIAALRKLKEQCGCRIYNLGTGTGYSVLQMVQAMEKASGKKIPYKVVARREGDVAAYYANPSLAQEELGWTAALGLDRMCEDLWRWQKQNPSGFGTQA

SEQ ID NO: 3 - DNA, 1017 bp, Eschenichia coli
atgagagttctggttaccggtggtagcggttacattggaagtcatacctgtgtgcaattactgcaaaacggtcatgatgtcatcattcttgataacctctgtaacagtaagcgcagcgtactgcctgttatcgagcgtttaggcggcaaacatccaacgtttgttgaaggcgatattcgtaacgaagcgttgatgaccgagatcctgcacgatcacgctatcgacaccgtgatccacttcgccgggctgaaagccgtgggcgaatcggtacaaaaaccgctggaatattacgacaacaatgtcaacggcactctgcgcctgattagcgccatgcgcgccgctaacgtcaaaaactttatttttagctcctccgccaccgtttatggcgatcagcccaaaattccatacgttgaaagcttcccgaccggcacaccgcaaagcccttacggcaaaagcaagctgatggtggaacagatcctcaccgatctgcaaaaagcccagccggactggagcattgccctgctgcgctacttcaacccggttggcgcgcatccgtcgggcgatatgggcgaagatccgcaaggcattccgaataacctgatgccatacatcgcccaggttgctgtaggccgtcgcgactcgctggcgatttttggtaacgattatccgaccgaagatggtactggcgtacgcgattacatccacgtaatggatctggcggacggtcacgtcgtggcgatggaaaaactggcgaacaagccaggcgtacacatctacaacctcggcgctggcgtaggcaacagcgtgctggacgtggttaatgccttcagcaaagcctgcggcaaaccggttaattatcattttgcaccgcgtcgcgagggcgaccttccggcctactgggcggacgccagcaaagccgaccgtgaactgaactggcgcgtaacgcgcacactcgatgaaatggcgcaggacacctggcactggcagtcacgccatccacagggatatcccgattaa

SEQ ID NO: 4 - protein, 338 aa, Eschesichia Coli
MRVLVTGGSGYIGSHTCVQLLQNGHDVIILDNLCNSKRSVLPVIERLGGKHPTFVEGDIRNEALMTEILHDHAIDTVIHFAGLKAVGESVQKPLEYYDNNVNGTLRLISAMRAANVKNFIFSSSATVYGDQPKIPYVESFPTGTPQSPYGKSKLMVEQILTDLQKAQPDWSIALLRYFNPVGAHPSGDMGEDPQGIPNNLMPYIAQVAVGRRDSLAIFGNDYPTEDGTGVRDYIHVMDLADGHVVAMEKLANKPGVHIYNLGAGVGNSVLDVVNAFSKACGKPVNYHFAPRREGDLPAYWADASKADRELNWRVTRTLDEMAQDTWHWQSRHPQGYPD

SEQ ID NO: 5 - DNA, 30 bp, Human DNA
ggtgatgtggcagcctattacgccaacccc

SEQ ID NO: 6 - DNA, 33 bp, Human DNA
gctggggttggcgtaataggctgccacatcacc

SEQ ID NO: 7 - DNA, 40 bp, Mouse DNA
cgcgacgcgtatccgtggcgggacgaatggacacagcaac

SEQ ID NO: 8 - DNA, 37 bp, Mouse DNA
cgcgaagcttatcggctccgctatgcgacgtgaggcc